(19) United States
(12) Patent Application Publication
(10) Pub. No.: US 2006/0019298 A1
(43) Pub. Date: Jan. 26, 2006

(54) Sequencing of modified nucleic acid molecules

(76) Inventors: David T. Shima, Boston, MA (US); Pericles Calias, Melrose, MA (US); Gregory S. Robinson, Wilmington, MA (US); Yin-Shan Ng, North Billerica, MA (US); Francine E. Wincott, Baltimore, MD (US); Corinna M. Krinos, Burlington, MA (US)

Correspondence Address: EYETECH PHARMACEUTICALS, INC., 3 TIMES SQUARE 12TH FLOOR, NEW YORK, NY, 10036, US

(21) Appl. No.: 11/184,240

(22) Filed: Jul. 19, 2005

Related U.S. Application Data
(60) Provisional application No. 60/590,601, filed on Jul. 23, 2004.

(51) Int. Cl. C12Q 1/68; C12P 19/34

(52) U.S. Cl. 435/6; 435/91.2

(57) ABSTRACT

Modified nucleic acids are used for many purposes in research, diagnostic, and treatment protocols. However, direct sequencing of such molecules is generally considered to require special conditions. Methods of making a cDNA using a modified nucleic acid molecule and sequencing methods of sequencing a modified nucleic acid molecule are described.

### RELATED APPLICATIONS

This Application claims the benefit of U.S. Provisional Application No. 60/590,601, filed on Jul. 23, 2004. The entire teachings of the above application is incorporated herein by reference.

### TECHNICAL FIELD

The application relates to the field of molecular biology. More specifically, the invention is directed to the sequencing of modified nucleic acids.

### BACKGROUND

Novel nucleic acid molecules such as ribozymes, aptamers, and siRNA (short interfering RNA) have various uses including elucidation of structural requirements, diagnostic applications, and therapeutic methods. In many cases, such nucleic acid molecules have one or more modifications of the nucleobase, sugar moiety and/or internucleoside linkage (Verma et al., 1998, Ann. Rev. Biochem. 67:99-134). Numerous modified oligonucleotides and nucleoside triphosphates are known and are available (for example, TriLink BioTechnologies, San Diego, Calif.; Lee et al., 2004, Nuc. Acids. Res. 32:Database issue D95-D100). However, one problem encountered with the use of modified nucleic acids is confirming the sequence of such molecules. Although nucleic acid molecules containing certain modifications have been sequenced, the sequencing of nucleic acid molecules with some types of modifications has been difficult or thought not to be possible using routine methods. For example, modified RNA sequences such as those conjugated to polyethylene glycol (PEG) cannot be sequenced using certain chemical degradation or mass spectrometry methods. Furthermore, difficulties have been encountered in sequencing nucleic acids containing multiple types of modifications, requiring the development of alternative techniques (see, e.g., Grosjean et al., 2004, Methods Mol. Biol. 265:357-91). Limitations have been described for the incorporation of non-natural nucleotides into oligonucleotides using various polymerases (Verma et al., 1998, supra; Kiss and Jady, 2004, Methods Mol Biol. 265:393-408).

Modified nucleic acid molecules are being employed in increasing numbers of uses, for example in diagnostic methods, screening methods (e.g., to identify pharmaceutical agents), and in the treatment of disease. For example, aptamers are nucleic acid molecules having a tertiary structure that permits them to specifically bind to protein ligands (e.g., Osborne, et al., 1997, Curr. Opin. Chem. Biol. 1:5-9; and Patel, 1997, Curr. Opin. Chem. Biol. 1:32-46). Such molecules can be selected from libraries of nucleic acids containing modified bases (e.g., using Systematic Evolution of Ligands by Exponential enrichment (SELEX™)). In some cases, a selected nucleic acid such as an aptamer is, subsequent to selection, chemically modified (for example, by methylation) and/or conjugated to, e.g., a PEG, lipid, lipoprotein, or liposome. Although the sequence of the original modified nucleic acid may be known, it is desirable to confirm the nucleic acid sequence after modification. For example, it may be desirable or even required that the nucleic acid sequence of the molecule be confirmed in a manufacturing protocol or for a drug approval process. However, there is evidence and belief in the art that certain types of modifications preclude sequencing modified nucleic acid molecules.

In general, modified nucleic acid molecules such as aptamers, siRNA, antisense nucleic acids, and ribozymes have not been sequenced using standard chemical degradation methods, while enzymatic methods of sequencing have been viewed as ineffective or require special conditions to sequence at least certain modified nucleic acids. In particular, enzymatic sequencing of nucleic acid molecules conjugated to PEG (i.e., pegylated) has not generally been undertaken.

### SUMMARY OF INVENTION

The invention relates to methods of sequencing a modified nucleic acid by synthesizing a cDNA complementary to a modified nucleic acid molecule. It has been discovered that a certain classes of modified nucleic acid molecules, containing one or more modifications such as methylation or pegylation, can be reverse transcribed, cloned, and sequenced. In some cases the nucleic acid molecule includes two or more types of modification, e.g., 2-fluoro substitution, 2-O-methyl substitution, and pegylation.

Thus, in one aspect, the invention provides methods for determining the nucleotide sequence of a modified nucleic acid which includes a multiplicity of 2′-modified nucleotides. These methods include the steps of (a) obtaining a sample of the modified nucleic acid; (b) synthesizing a first cDNA complementary to the modified nucleic acid using a reverse transcriptase; (c) synthesizing a second cDNA complementary to the first cDNA using a DNA polymerase to form a double-stranded cDNA; (d) producing multiple double-stranded copies of the double-stranded cDNA; and (e) sequencing the double-stranded copies. In these methods, the modified nucleic acids include a multiplicity of 2′-fluorinated nucleotides and a multiplicity of 2′-O-methylated nucleotides.

In some embodiments, the 2′-fluorinated nucleotides comprise between 10% and 70% of the total nucleotides in the modified nucleic acid. In some embodiments, the 2′-fluorinated nucleotides comprise at least 40% of the total nucleotides in the modified nucleic acid.

In some embodiments, the 2′-O-methylated nucleotides comprise between 10% and 70% of the total nucleotides in the modified nucleic acid. In some embodiments, the 2′-O-methylated nucleotides comprise at least 40% of the total nucleotides in the modified nucleic acid.

In some embodiments, the 2′-fluorinated nucleotides comprise between 10% and 70% of the total nucleotides in the modified nucleic acid and the 2′-O-methylated nucleotides comprise between 10% and 70% of the total nucleotides in the modified nucleic acid.

In some of the foregoing embodiments, the modified nucleic acid further includes a 5′ covalent modification including a large hydrophilic moiety.

In another aspect, the invention provides methods for determining the nucleotide sequence of a modified nucleic acid including a 5′ or 3′ large hydrophilic moiety. These methods include the steps of (a) obtaining a sample of the modified nucleic acid; (b) synthesizing a first cDNA complementary to the modified nucleic acid using a reverse transcriptase; (c) synthesizing a second cDNA complementary to the first cDNA using a DNA polymerase to form a double-stranded cDNA; (d) producing multiple double-stranded copies of the double-stranded cDNA; and (e) sequencing the double-stranded copies.

In some embodiments, the large hydrophilic moiety is selected from a branched or straight-chain, substituted or unsubstituted, homopolymer or heteropolymer of alkyl, alkenyl, aryl, or heterocyclic groups. In some embodiments, the large hydrophilic moiety is a polyethylene glycol moiety, such as a 10-50 kDa polyethylene glycol moiety.

In any of the foregoing embodiments including a large hydrophilic moiety, the modified nucleic acid can further include at least one 2′-fluorinated nucleotide or at least one 2′-O-methylated nucleotide.

In other embodiments, the invention provides methods for determining the nucleotide sequence of a modified nucleic acid which includes a multiplicity of 2′-modified nucleotides. These methods include the steps of (a) obtaining a sample of the modified nucleic acid; (b) synthesizing a first cDNA complementary to the modified nucleic acid using a reverse transcriptase; (c) purifying the first cDNA; (d) polyadenylating the 3′-end of the first cDNA; (e) synthesizing a second cDNA complementary to the first cDNA using a DNA polymerase to form a double-stranded cDNA; (f) producing multiple double-stranded copies of the double-stranded cDNA; and (g) sequencing the double-stranded copies. In these methods, the modified nucleic acids include a multiplicity of 2′-fluorinated nucleotides and a multiplicity of 2′-O-methylated nucleotides.

In some embodiments of the foregoing methods, the step of producing multiple double-stranded copies of the double-stranded cDNA includes the steps of (i) ligating the double-stranded cDNA into a cloning vector; (ii) transforming host cells with the cloning vector; (iii) isolating the cloning vector from descendants of the host cells; and (iv) isolating the double-stranded copies from the host cells.

In other embodiments of the foregoing methods, the step of producing multiple double-stranded copies of the double-stranded cDNA includes performing the polymerase chain reaction using the double-stranded cDNA as an original template molecule.

In another aspect, the invention provides methods of synthesizing a DNA complementary to a modified nucleic acid which includes a multiplicity of 2′-modified nucleotides. These methods include the steps of (a) obtaining a sample of the modified nucleic acid; and (b) synthesizing a first cDNA complementary to the modified nucleic acid using a reverse transcriptase. In these methods, the modified nucleic acid includes a multiplicity of 2′-fluorinated nucleotides and a multiplicity of 2′-O-methylated nucleotides.

In some embodiments, the modified nucleic acid further includes a 5′ covalent modification comprising a large hydrophilic moiety. In some of these embodiments, the large hydrophilic moiety is selected from a branched or straight-chain, substituted or unsubstituted, homopolymer or heteropolymer of alkyl, alkenyl, aryl, or heterocyclic groups. In certain embodiments, the large hydrophilic moiety includes a polyethylene glycol moiety, such as a 10-50 kDa polyethylene glycol moiety.

In some of the foregoing embodiments, the modified nucleic acid further includes a modified 3′ terminus.

Although methods and materials similar or equivalent to those described herein can be used in the practice or testing of the present invention, suitable methods and materials are described below. In addition, the materials, methods, and examples are illustrative only and not intended to be limiting.

Other features and advantages of the invention will be apparent from the detailed description, drawings and claims.

### DETAILED DESCRIPTION

All publications, patent applications, patents, and other references mentioned herein are incorporated by reference in their entirety.

Unless otherwise defined, all technical and scientific terms used herein have the same meaning as commonly understood by one of ordinary skill in the art to which this invention belongs.

As used herein, unless specifically indicated otherwise, the word “or” is used in the “inclusive” sense of “and/or” and not the “exclusive” sense of “either/or.”

As used herein, the recitation of a numerical range for a variable is intended to convey that the invention may be practiced with the variable equal to any of the values within that range. Thus, for a variable that is inherently discrete, the variable can be equal to any integer value within the numerical range, including the end-points of the range. Similarly, for a variable that is inherently continuous, the variable can be equal to any real value within the numerical range, including the end-points of the range. As an example, and without limitation, a variable that is described as having values between 0 and 2 can take the values 0, 1, or 2 if the variable is inherently discrete, and can take the values 0.0, 0.1, 0.01, 0.001, or any other real values ≧0 and ≦2 if the variable is inherently continuous.

A modified nucleic acid (MNA) is a nucleic acid molecule that contains either (a) at least one methylation (e.g., a 2-O-methyl substitution) and at least one 2′-fluoro substitution (e.g., a 2-fluoro-pyrimidine substitution), or (b) at least one 5′ or 3′ modification with a large hydrophilic moiety. An MNA can include one of more additional types of modifications or substitutions.

The methods described and claimed herein are useful for generating cDNA complementary to an MNA sequence, and for cloning and sequencing an MNA molecule.

Reverse Transcription of Modified Nucleic Acid Molecules

Synthesis of a cDNA by reverse transcription of an MNA is performed by first identifying an MNA to be reverse transcribed. For example, the MNA can be a molecule for which the sequence is to be confirmed after scaling up production, in studies of shelf life, or other protocols requiring sequence information. It is understood that, as described herein, a cDNA sequence prepared from an MNA will not contain the modifications of the MNA but will contain the naturally occurring nucleotides that correspond to the modified nucleotides of the MNA. In the present methods, a DNA oligonucleotide primer complementary to the 3′ terminus of the MNA is prepared using methods known in the art such as chemical synthesis. In general, if the 3′ sequence contains MNAs the primer is prepared using naturally occurring deoxynucleotide triphosphates that are complementary to the naturally occurring nucleotides that most closely correspond to the portion of the MNA sequence targeted by the primer. To facilitate cDNA synthesis, a synthetic sequence such as a polyA tail can be added to the 3′ terminus of the MNA, or a predetermined sequence can be ligated to the 3′ end of the MNA. Such methods are generally not employed when the ultimate 3′ nucleotide of the MNA is modified.

The primer can be any length which results is sufficient sequence-specific annealing under the chosen reaction conditions. Typically, primers will be 5-20 nucleotides in length, with reverse transcriptases employing shorter sequences (e.g., 5-10 nucleotides) and DNA polymerases employing longer sequences (e.g., 12-18 nucleotides). Generally, longer primers results in higher efficiency and specificity of annealing. The primer is incubated with the MNA and a cDNA complementary to the MNA is synthesized using reverse transcriptase (RT) and deoxynucleotide triphosphates (dNTPs). The reverse transcriptase can be from any source, for example, avian myeloblastosis virus, Moloney murine leukemia virus, or an engineered RT that, for example, lacks RNAse H activity or has other features (e.g., OmniScript™ RT, Qiagen, Valencia, Calif.; SuperScript™ II RNase H, Invitrogen). The dNTPs used in the reverse transcriptase reaction can be standard dNTPs or modified dNTPs (see, e.g., Krayevsky et al., 1998, Nucleosides Nucleotides 17(7):1153-62; Tasara et al., 2003, Nucleic Acids Res. 31(10):2636-46). In general, if the MNA is to be sequenced, naturally occurring dNTPs (dCTP, dATP, dTTP, and dGTP) are used for the RT reaction. Reagents for the reverse transcriptase reaction are known in the art and are available from commercial sources. The reverse transcription reaction can be performed according to known protocols appropriate for the specific RT used in the reaction. However, such protocols can include modifications suggested by the manufacturer, known to those of skill in the art, or developed by routine experimentation for use with the particular MNA and RT.

Optimization of Reverse Transcription

Several parameters of the reverse transcription reaction can be manipulated to increase the fidelity and amount of cDNA produced when using an MNA template. The amount of MNA used can be at least 10 pmoles, 20 pmoles, 50 pmoles, or 100 pmoles per reaction volume. For example the amount of MNA used can be 1-100 pmoles, 10-100 pmoles, or 50-100 pmoles per reaction volume. The amount of template can be at least 10 pmoles, 20 pmoles, 50 pmoles, or 100 pmoles per reaction volume. For example, the amount of template used can be about 1-100 pmoles, 10-100 pmoles, or 50-100 pmoles per reaction volume. The amount of primer used can be at least 25, 50, 75, 100, 125, 150, 250, or 300 pmoles per reaction volume. For example, the amount of primer template used can be about 10-300 pmoles, 25-300 pmoles, 125-300 pmoles, 150-300 pmoles, or 250-300 pmoles per reaction volume. The reaction volumes can vary depending upon the quantities of starting materials available and the amount of final product desired. For example, reaction volumes can be 5-50 μl (for example, 12 μl for primer annealing and 20 μl for the RT reaction). In general, equal amounts of each dNTP are used in the reverse transcriptase reaction. In some cases, the concentration of each dNTP in the reaction is about 500 μM, 600 μM, 700 μM, 750 μM, 1,000 μM, 1,250 μM, or 2,500 μM. For example, the concentration can be from about 500 μM-2,500 μM, about 500 μM-1,000 μM, or from about 1,000 μM to about 2,500 μM. A tracer is used in certain reverse transcriptase reactions so that the cDNA product can be detected. In such cases, an α³²P-dNTP can be used (e.g., [α³²P]-dCTP). dNTPs that include other types of labels, such as Cy3- or Cy5-labeled nucleotides, can also be used. For radio-labeling, the amount of labeled dNTP is, for example, about 10 μCi, 20 μCi, or 25 μCi per reaction volume. For example, the amount of labeled dNTP can be from about 10 μCi-25 μCi, or from about 10 μCi-20 μCi per reaction volume.

Other parameters that can be varied are the reaction temperature and reaction time. Examples of temperatures and times used to reverse transcribe an MNA are generally at least 50 minutes at 42° C., or 55 minutes at 42° C., or 60 minutes at 42° C., followed by 15 minutes at 37° C., and 10 minutes at 70° C. for enzyme inactivation when using SuperScript™ II RNase H. For example, the temperature for a reverse transcription reaction can be from about 37° C.-50° C. (e.g., 42° C. for first strand synthesis with SuperScript™ II). The time for a reverse transcription reaction as used herein can be, for example, from about 30-60 minutes, about 40-60 minutes or about 50-60 minutes. Parameters can also be adjusted according to the manufacturer's recommendations for a specific reverse transcriptase. A general, nonlimiting example of a reverse transcription protocol for synthesizing a cDNA complementary to a selected MNA is as follows; suspend the MNA in water and heat to denature (e.g., for 2 minutes at 90° C.), cool the sample (e.g., on ice for 2 minutes), add the unlabeled dNTPs, primer and water heat at an appropriate temperature (e.g., 42° C.-70° C.) for primer annealing (e.g., for 5 minutes), cool, add first strand buffer, DTT, labeled dNTP, and the reverse transcriptase enzyme, incubate the reaction mixture at room temperature (e.g., for 10 minutes) then at a higher temperature (e.g., at 42° C.) for 40-60 minutes, followed by incubation for 15 minutes at 37° C. and 10 minutes at 70° C. The sample can then be analyzed, for example, by adding gel loading buffer to the RT reaction mixture and electrophoresing. In some cases, denaturing gel conditions are used.

Processing and Sequencing

The first strand RT reaction product can be detected and isolated for additional processing and sequencing. For example, after first strand cDNA isolation (e.g., extraction and precipitation using phenol/chloroform and ethanol), the second strand of cDNA can be obtained using an appropriate primer and PCR can be used to amplify the double-stranded cDNA. If the 3′ sequence of the first strand cDNA ids not known, a known sequence can be ligated to the 3′ end, and a primer complementary to the known sequence can be employed. Alternatively, a polyA tail can be added to the first strand using terminal deoxynucleotide transferase, and an oligo dT primer can be used for second strand synthesis. For cloning, the resulting double-stranded DNA can be ligated into a cloning vector, transformed into bacteria, and bacteria containing the cloned sequences are identified. Sequences amplified by PCR or cloning are then isolated and sequenced using known protocols.

Modified Nucleic Acid Molecules

The MNA molecules that can be sequenced using the methods described herein include modified RNA, modified DNA, aptamer, ribozyme, and siRNA molecules. Such molecules can contain synthetic nucleic acid analogs or a combination of naturally occurring and synthetic nucleic acid analogs. Synthetic nucleic acid analogs include those containing one or more backbone, base, or sugar modifications. Such modifications are known in the art, for example, see Verma et al. (Ann. Rev. Biochem. (1998) 67:99-134). In particular, MNA molecules that can be sequenced using these methods include nucleic acid molecules containing modifications such as 2′-fluoro substitution (e.g., 2′-fluoro-pyrimidine or 2′-fluoropurine), 2′-O-methyl substitution, pegylation (e.g., by covalent attachment of a PEG at the 5′ terminus), an inverted deoxythymidine cap, or other modifications of the 5′ or 3′ ends. Modified nucleic acid molecules can contain one of more modifications. For example, the molecule can contain both 2′-fluoropyrimidine and 2′-O-methylpurine substitutions, and optionally, a PEG moiety.

In some cases the MNA molecule is an antisense ribonucleic acid. An “antisense” nucleic acid is a nucleotide sequence that is complementary to a “sense” nucleic acid encoding a protein, e.g., complementary to the coding strand of a double-stranded cDNA molecule, to an mRNA sequence or to a transcribed non-coding sequence (e.g., the 5′ and 3′ untranslated regions of a gene). The antisense nucleic acid can be complementary to an entire coding strand, or to only a portion thereof. One nonlimiting example of an antisense molecule is a ribozyme, which is a catalytic nucleic acid molecule. Antisense molecules and other nucleic acid molecules can be chemically synthesized using naturally-occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The methods described herein can be used to sequence such modified molecules.

Nucleic acids containing other types of modifications can also be reverse transcribed, cloned, and sequenced as described herein. For example, for systemic administration, ribonucleic acid molecules can be modified such that they specifically bind to receptors or antigens expressed on a selected cell surface, e.g., by linking the ribonucleic acid molecule to a peptide or antibody that binds to cell surface receptors or antigens. Additional examples of MNA molecules include those containing one or more 2′-O-methylnucleotides (Inoue et al., 1987, Nucleic Acids Res. 15:6131-6148) or chimeric RNA-DNA analogs (Inoue et al., 1987, FEBS Lett. 215:327-330). Nucleic acid molecules containing detectable labels (e.g., fluorescent, chemiluminescent, radioactive, or colorimetric) can be reverse transcribed, cloned, and sequenced using the described methods.

In some cases, the MNA molecule includes a large hydrophilic moiety. Generally, such moieties are covalently attached to the nucleic acid molecule. Large hydrophilic moieties include branched or straight chain, substituted or unsubstituted, homopolymers or heteropolymers of alkyl, alkenyl, aryl, or heterocyclic groups. Specific examples include polyethylene glycol (e.g., 10-50 kDa PEG, 20-40 kDa PEG), and other non-nucleic acid molecules such as dextrans, carboxymethylcellulose or polyHEMA (i.e., poly(2-hydroxyethylmethacrylate)).

Such large hydrophilic moieties can be conjugated to the MNA at any position on the nucleic acid sequence. (See, for example, U.S. application Ser. No. 60/561,601, the entire disclosure of which is incorporated by reference herein.) For example, conjugation of the large hydrophilic moiety can be through the 5′ end of the MNA, the 3′ end of the MNA, or any position along the MNA sequence between the 5′ and 3′ ends. For example, the large hydrophilic moiety can be conjugated to the MNA at an exocyclic amino group on a base, a 5-position of a pyrimidine nucleotide, an 8-position of a purine nucleotide, a hydroxyl group of a phosphate, or a hydroxyl group of a ribose group of the modified nucleic acid sequence. Means for chemically linking large hydrophilic moieties to MNA sequences at these various positions are known in the art and/or exemplified below.

Examples of large hydrophilic moieties include polymers (e.g., polyethylene glycol), gel-forming compounds and the like. Examples of particularly useful large hydrophilic moieties include polyethylene glycols, polysaccharides, such as glycosaminoglycans, hyaluronans, and alginates, polyesters, high molecular weight polyoxyalkylene ether (such as PluronicTM), polyamides, polyurethanes, polysiloxanes, polyacrylates, polyols, polyvinylpyrrolidones, polyvinyl alcohols, polyanhydrides, carboxymethyl celluloses, other cellulose derivatives, chitosan, polyadlehydes or polyethers. Particularly useful large hydrophilic moieties will have a molecular weight of from about a molecular weight of about 20 to about 100 kDa.

Furthermore, the addition of non-immunogenic, high molecular weight or lipophilic compounds to the 5′ end, to improve nuclease resistance and/or other pharmacokinetic properties, has also been described (see, for example, U.S. Pat. No. 6,011,020, U.S. Pat. No. 6,147,024, U.S. Pat. No. 6,229,002, U.S. Pat. No. 6,426,335, U.S. Pat. No. 6,465,188, and U.S. Pat. No. 6,582,918), and such groups can be employed as large hydrophilic moieties. Examples of lipophilic groups are saturated or unsaturated hydrocarbons such as alkyl, alkenyl or other lipid groups. Sterols (e.g., cholesterol) and other pharmaceutically acceptable adjuvants (including anti-oxidants like alpha-tocopherol) can also be included. In general, such “lipophilic compounds” are compounds which have the propensity to associate with or partition into lipid and/or other materials or phases with low dielectric constants, including structures that are comprised substantially of lipophilic components. Lipophilic compounds include lipids as well as non-lipid containing compounds that have the propensity to associate with lipid (and/or other materials or phases with low dielectric constants). Cholesterol, phospholipids, and glycerolipids, such as dialkylglycerol, and diacylglycerol, and glycerol amide lipids are further examples of such lipophilic compounds.

In some cases, the MNAs include appended groups such as peptides (e.g., for targeting host cell receptors in vivo), or agents facilitating transport across the cell membrane (see, e.g., Letsinger et al., 1989, Proc. Natl. Acad. Sci. (USA) 86:6553-6556; Lemaitre et al., 1987, Proc. Natl. Acad. Sci. (USA) 84:648-652; PCT Publication No. WO88/09810) or the blood-brain barrier (see, e.g., PCT Publication No. WO 89/10134). In addition, oligonucleotides can be modified with hybridization-triggered cleavage agents (see, e.g., Krol et al., 1988, Bio-Techniques 6:958-976) or intercalating agents (see, e.g., Zon, 1988, Pharm. Res. 5:539-549). To this end, the oligonucleotide can be conjugated to another molecule (e.g., a peptide, hybridization triggered cross-linking agent, transport agent, or hybridization-triggered cleavage agent). Yet another type of MNA that can be sequenced using the methods described herein are molecular beacon oligonucleotide primer and probe molecules that have at least one region that is complementary to a selected nucleic acid, and two complementary regions, one of which has a fluorophore and one of which has a quencher, such that the molecular beacon is useful for quantitating the presence of the selected nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Vascular endothelial growth factor (VEGF) plays a role in angiogenesis and exists in several isoforms. VEGF₁₆₅ is the most abundant isoform in humans and is an angiogenic cytokine that is also involved in aberrant angiogenesis and vascular permeability and glomerular endothelial repair (Ostendorf et al., 1999, J. Clin. Investigation 104:913-923). Aptamers (from the Greek aptus—to fit—and meros—part or region) are oligonucleotides that bind with very high specificity and affinity to target molecules, including proteins. A 28-nucleotide aptamer that contains 2′ fluoropyrimidines and specifically blocks VEGF₁₆₅ binding to the FLT-1 and KDR VEGF receptors was prepared and identified using systematic evolution of ligands by exponential enrichment (SELEX™, Gilead Sciences, Inc., Foster City, Calif.). The aptamer was further modified by 2′-O-methyl substitutions of all but two purine nucleotides, by addition of two branched 20 kDa PEG moieties conjugated to the 5′ terminus of the aptamer, and by linkage of a deoxythymidine to the 3′ terminus via a 3′-3′ linkage (Ruckman et al., 1998, J. Biol. Chem. 273:20556). This elaborately modified aptamer (termed NX1838 or Pegaptanib) is being used in human clinical testing.

Pegaptanib, which contains a diversity of modifications, was used as a test molecule for identifying methods of determining the nucleic acid sequence of molecules containing modifications that may otherwise be considered to render the molecule unsuitable for reverse transcription and sequencing. The Examples illustrate cDNA synthesis and cloning of this MNA molecule.

### EXAMPLES

The invention is further illustrated by the following examples. The examples are provided for illustrative purposes only. They are not to be construed as limiting the scope or content of the invention in any way.

### Example 1

### Base Sequence Determination of a Pegylated Aptamer

The following procedures were used to determine the base sequence of the 2′-fluoropyrimidine, 2′-O-methylpurine, 5′ pegylated aptamer (Pegaptanib) via reverse transcription (cDNA synthesis), followed by cloning and DNA sequencing.

The synthesis of Pegaptanib is described in Ruckman et al. 1998, J. Biol. Chem. 273:20556. The concentration of Pegaptanib was determined using a spectrophotometer to measure the UV absorbance of diluted Pegaptanib samples at 260 nm (OD₂₆₀) using the following equation (Beer-Lambert law):
C (mg/ml)=[A/(e×b)]×(dilution factor)
C=concentration; A=(OD₂₆₀); e=extinction coefficient=27.29; b=1
   First Strand cDNA Synthesis Via Reverse Transcription

Conditions were determined for synthesis of a cDNA for the modified nucleic acid Pegaptanib. In general, a primer (termed MACRT1) was used for synthesizing a cDNA using reverse transcriptase. The primer, which had the sequence (5′ to 3′) CGGATGTA, was synthesized by Invitrogen Life Technologies, Inc. First strand synthesis was performed using 200 units Superscript™ II RNaseH- Reverse Transcriptase (Invitrogen, Inc., cat. # 18064-022), 5× first strand buffer and 0.1 M DTT (dithiothreitol; Invitrogen Inc., supplied with RT enzyme), dNTP mix containing 10 mM of each deoxyribonucleotide (Invitrogen, Inc., cat. # 18427-088), [α³²P]-dCTP (PerkinElmer Life Sciences, Inc., cat. # BLU013H), nuclease-free water (Ambion, Inc., cat. # 9937), and RNAse- DNAse-free microcentrifuge tubes (Ambion, Inc., cat. # 12450).

To determine an appropriate amount of Macugen template to use in the RT reaction, various concentrations of Pegaptanib were used in an RT reaction; 10 pmoles, 20 pmoles, 50 pmoles, and 100 pmoles. In addition, different concentrations of primer were used in the reactions; 25 and 50 pmoles of primer with 10 pmoles of template, 50 pmoles and 60 pmoles of primer with 20 pmoles of template; 50, 125, and 150 pmoles of primer with 50 picomoles of template; and 50, 250, and 300 pmoles of primer with 100 pmoles of template. All reactions were performed in a final volume of 20-25 μl. The molar equivalents of primer used in the RT reactions were 50 pmoles˜2.5 μM, 60 pmoles˜3.0 μM, 125 pmoles˜6.25 μM, 150 pmoles˜7.5 μM, 250 pmoles˜12.5 μM, and 300 pmoles˜15 μM.

In another set of reactions designed to characterize the RT reaction of the MNA, various concentrations of dNTPs were tested. The tested concentrations were 10 pmoles template with 500 μM of each dNTP; 20 pmoles template with 500 μM or 550 μM of each dNTP; 50 pmoles template with 700 μM, 750 μM, 1,000 μM, or 1250 μM of each dNTP; and 100 pmoles template with 750 μM, 1,000 μM, 1,250 μM, or 2,500 μM of each dNTP.

In an additional set of experiments designed to assess conditions for the RT reaction, various amounts of [α-³²P]-dCTP (e.g., 10 μCi, 20 μCi, and 25 μCi) were tested in the RT reactions. The assays were performed in a checkerboard format in which different primer and template amounts were tested. In one of these experiments, the optimal conditions for RT reactions with the MNA Pegaptanib included 20-25 μCi of [α-³²P]-dCTP with 10 pmole template and 50 pmole primer in a reaction volume of 20-25 μl.

The following general protocol was used for each of the experiments described above. Template (Pegaptanib) was suspended in water and heated at 90° C. for 2 minutes and then cooled on ice for 2 minutes. The dNTPs and primer were added to the template and the mixture was heated for 5 minutes at 65° C., then cooled on ice for 2 minutes, followed by addition of the [α³²P]-dCTP and incubation for an additional 5 minutes at 42° C. Next, 200 units of Superscript™ II RNaseH was added to the reaction mixture, which was then incubated for 10 minutes at room temperature followed by 60 minutes incubation at 42° C. This reaction mixture was incubated for an additional 15 minutes at 37° C., and the reaction terminated by adding gel loading buffer. Samples were analyzed by electrophoresis through a 15% polyacrylamide gel containing 8M urea. The reaction products were detected by exposure of the gel to film for one to 5 hours or overnight.

Conditions were selected by identifying the conditions that resulted in the production of a correct sized cDNA product and maximal [α³²P]-dCTP incorporation compared to other samples. After testing a large variety of reaction conditions in which template, primer, dNTP and radionuclide concentrations were varied, conditions were selected which yielded consistent reproducible results.

Conditions for Reverse Transcription of Pegaptanib

It was determined that, of the conditions tested, the best starting concentration of Pegaptanib was 10 pmoles in a reaction containing 50 pmoles of primer (MACRT1), 500 μM dNTPs, and which was incubated for 60 minutes at 42° C. Accordingly, one useful protocol for the synthesis of a cDNA to the MNA molecule Pegaptanib was found to be as follows. Pegaptanib was heated at 90° C. for 2 minutes and cooled on ice for 2 minutes. The following reagents were then added to an appropriate reaction container in indicated order to make an initial reaction mixture; a volume of water that brought the total volume to 12 μl, dNTPs (500 μm of each dNTP), MACRT1 primer (50 pmoles), and Pegaptanib (10 pmoles). The initial reaction mixture was then incubated at 65° C. for 5 minutes and cooled on ice for 2 minutes. Next, 4 μl of 5× first strand buffer, 2 μl of DTT (0.1M), and 2.5 μl (25 μCi) [α³²P]-dCTP was added, centrifuged briefly, incubated for 2 minutes at 42° C., and then 1 μl of SuperScript™ II RNAse H- reverse transcriptase was added to make a final reaction mixture. The final reaction mixture was incubated for 10 minutes at 25° C., then for one hour at 42° C., followed by 37° C. for 10 minutes. The reaction was terminated by adding an equal volume of Gel Loading Buffer II to the final reaction mixture. The RT cDNA samples were then frozen for future use or analyzed by electrophoresis, and, if desired, purified.

Purification of First Strand cDNA

Analysis of a cDNA prepared from Pegaptanib was analyzed and purified using polyacrylamide gel electrophoresis. The following reagents were used for PAGE resolution of RT products; 40% acrylamide/bis (19:1) solution (Ambion, Inc., cat. # 9022), 10× TBE buffer (Ambion, Inc., cat. # 9863), urea (ultrapure molecular biology grade; Ambion, Inc., cat. # 9900), ammonium persulfate (10% solution in water, Sigma, Inc., cat. # A-3678), N,N,N′,N′-tetramethylethylenediamine (TEMED) (Sigma, Inc., cat. # T-8133), Gel loading buffer II (denaturing PAGE, Ambion, Inc., cat. # 7140), RNA Decade Size Markers (Ambion, Inc., cat. # 7778), 1 M Tris pH 8.0 (Ambion, Inc., cat. # 9855G), 0.5 M EDTA pH 8.0 (Ambion, Inc., cat. # 9260G), 3 M sodium acetate, pH 5.5 (Ambion, Inc., cat. # 9740), Tris saturated phenol, pH 8 (Ambion, Inc., cat. # 9710), and chloroform:isoamyl alcohol (24:1) mix (Sigma, Inc., cat. # C-0549).

Briefly, all or a portion of an RT cDNA sample was loaded onto a 15% polyacrylamide gel containing 8 M urea and 1× TBE with a Decade Marker System that was radiolabeled with γ³²P-ATP as a marker. The gel was run for one hour at 120 volts and exposed to X-ray film (either for 4 hours at −70° C. or overnight) to determine position of first strand cDNA products. Gel slices containing the desired products were excised, cut into small pieces and eluted in 200 μl extraction buffer (1 mM Tris, 0.5 mM EDTA, pH 8) with shaking overnight at 37° C. The first eluate was collected and an additional 200 μl of extraction buffer added to the gel pieces, which were then incubated for 2 hours at 37° C. The second eluate was combined with the first and sodium acetate was added to the eluates to a final concentration 0.3 M. The eluates, which contained the cDNA, were extracted with an equal volume of Tris-saturated phenol:chloroform (containing isoamyl alcohol) (1:1) mix, then with chloroform containing isoamyl alcohol, and glycogen was added to a final concentration of 100 μg/ml. The cDNA was precipitated in three volumes of ethanol either on dry ice for 2 hours or overnight at −80° C. The precipitated cDNA was collected by centrifugation at 20,000× g for 15 minutes. The ethanol was removed, the pellet washed once with 70% ethanol, air dried for about 10 minutes, and resuspended in 20 μl water.

PolyA Tailing of Purified First Strand cDNA

To prepare the purified Pegaptanib cDNA for additional procedures, the cDNA was 3′ polyadenylated using terminal deoxynucleotide transferase (TdT). In general, the goal was to add a polyA tail of about 15-25 nucleotides to the cDNA. The reagents used in the reaction included terminal transferase (TdT) (Stratagene cat. # 600137), buffer (supplied with TdT enzyme), dATP (25 mM, Invitrogen, Inc., cat. # 18427-013), and Centri-Spin™-10 size exclusion spin columns (Princeton Separations, Inc., cat. # CS-101). Briefly, 2 pmoles of cDNA was used in the polyadenylation reaction (about 10 μl of the resuspended cDNA described supra). One μll dATP (final concentration 0.5 μM), 10 μl 5× tailing buffer, and 0.5 μl (10 units) TdT were added to the cDNA. The final volume of the reaction was 50 μl. In some experiments, the dATP was [α³²P]-dATP. The polyadenylation reaction mixture was then briefly centrifuged. Various incubation times for the polyadenylation reaction were tested: 5, 10, 15, and 20 minutes using cDNA samples prepared as described above using 10 pmoles, 20 pmoles, 50 pmoles, or 100 pmoles Pegaptanib as the template. Incubations were at 37° C. followed by heat treatment for 10 minutes at 70° C. to inactivate the TdT. The entire sample was then column purified using a Centri-Spin™-10 exclusion spin column.

For samples that were to be cloned and sequenced, an incubation time of 6 minutes was selected for the TdT reaction with Pegaptanib cDNA to obtain a polyA tail of suitable length.

Second Strand cDNA Synthesis

To prepare a double-stranded nucleic acid from a Pegaptanib cDNA, second strand synthesis was performed and the resulting double-stranded DNA amplified using the polymerase chain reaction (PCR). The following reagents were used; PfuTurbo® DNA Polymerase (Stratagene, Inc., cat. # 600250), 10× pfu buffer (Stratagene, Inc., supplied with the PfuTurbo® enzyme), dNTP mix containing 10 mM of each deoxyribonucleotide (Invitrogen, Inc., cat. # 18427-088), and poly-T (oligo-dT) primer (16 mer, Roche, Inc., Lot # E00705). Briefly, second strand synthesis was carried out using column-purified single-stranded Pegaptanib cDNA containing a polyA tail as a template. In general, the entire column-purified product from a single polyadenylation reaction was used (about 50 μl, which contains about 1.2 pmoles/50 μl; cDNA is present at about 0.024 pmole/μl). The following were added to 50 μl of sample: 1 μdNTPs, 5 μl 10× pfu buffer, 0.5 μl oligo-dT primer, and 0.55 μl PfuTurbo® DNA polymerase. This second strand synthesis reaction was incubated at 95° C. for 1.15 minutes, 42° C. for 1 minute, then 75° C. for 10 minutes, followed by incubation at 4° C. for 5 minutes in a DNA Engine Tetrad PCR machine for a single cycle (PTC-225 Peltier Thermal Cycler, MJ Research, Waltham, Mass.).

Blunting and 5′-End Phosphorylation of the Double-Stranded cDNA

To prepare the double-stranded Pegaptanib for subcloning, the double-stranded DNA was blunt ended and the blunt ends phosphorylated. Blunt ending was performed using the following reagents: T4 DNA polymerase (New England Biolabs, Inc., cat. # M0203L), 10× T4 DNA polymerase buffer (New England Biolabs, Inc., supplied with T4 DNA polymerase enzyme), bovine serum albumin (BSA, 10 mg/ml, New England Biolabs, Inc., supplied with T4 DNA polymerase enzyme), and dNTP mix containing 10 mM of each deoxyribonucleotide (Invitrogen, Inc., cat. # 18427-088). The blunt end reaction mixture contained 0.5 μl of dNTP (10 μM each dNTP), 0.5 μl of 100× BSA (10 mg/ml), 5.5 μl of 10× buffer for T4 DNA polymerase, and 0.5 μl T4 DNA polymerase. The reaction mixture was incubated at 12° C. for 20 minutes, 75° C. for 10 minutes followed by a 4° C. incubation for 5 minutes.

The blunt ended double-stranded nucleic acid was then phosphorylated at the 5′ termini using the following reagents: T4 polynucleotide kinase (PNTK) (New England Biolabs, Inc., cat. # M0201L), 10× T4 polynucleotide kinase buffer (New England Biolabs, Inc., supplied with PNTK enzyme), and ATP solution, 10 mM (Ambion, Inc., cat. # 8110G). The phosphorylation reaction was performed in the same container as was used for the blunt end reaction by adding 6 μl 10× PNTK buffer, 3 μl ATP solution, and 1 μl PNTK enzyme. The reaction mixture was incubated at 37° C. for 60 minutes, 65° C. for 20 minutes, then stored at 4° C. or immediately followed by DNA extraction.

DNA Extraction

To extract the double-stranded DNA that was prepared as described above, the volume of the reaction mixture from the phosphorylation step was brought to 200 μl with nuclease-free water. Next, 20 μl of a 3 M sodium acetate solution was added followed by 100 μl phenol/Tris solution and vigorous mixing, then 100 μl chloroform and vigorous mixing. The solution was centrifuged for 10 minutes to separate the phases, the aqueous phase was collected and re-extracted with 100 μl chloroform, followed by centrifugation for 10 minutes, collection of the aqueous phase, and then addition of 4 μl glycogen (5 mg/ml stock solution). Next, 3 volumes of 95% ethanol were added and DNA was precipitated either overnight at −80° C. or on dry ice for 2 hours. Following the precipitation step, the cDNA was centrifuged for 15 minutes, the ethanol removed, and the pellet washed once with 70% ethanol. The pellet was briefly air dried and resuspended in 10 μl of nuclease-free water.

Ligation of cDNA to pBluescript

The double-stranded Pegaptanib-derived nucleic acids that were prepared as described above were then cloned into pBluescript II SK (+) that was linearized by EcoRV and purified by agarose gel electrophoresis. The EcoRV linearized pBluescript vector contains blunt ends suitable for ligation with double-stranded cDNA that contains blunt ends. The reagents used for the cloning protocol were as follows: plasmid vector pBluescript® II SK (+) (EcoRV V digested and purified; Stratagene, Inc., cat. # 212205), Quick Ligation™ Kit (supplied with T4 DNA ligase and 10× Quick Ligation buffer) (New England Biolabs, Inc., cat. # M2200S), XL1-Blue Electroporation-Competent Cells (Stratagene, Inc., cat. # 200228), SOC Medium (Invitrogen, Inc., cat. # 15544-034), Gene Pulser® Cuvette (Bio-Rad Laboratories, cat. # 165-2089), Brain Heart Infusion growth medium (Becton Dickinson, Inc., cat. # 237500), ampicillin Sodium Salt, 0.1 mg/ml in water (Calbiochem, Inc. cat. # 171254), Difco LB agar (Becton Dickinson, Inc., cat. # 240110), restriction endonucleases EcoRV V, Xba I, and Xho I (New England Biolabs, Inc., cat. # R0195S, R0146S, R0145S, respectively), QIAprep® Spin Miniprep DNA Kit (Qiagen, Inc., cat. # 27104), agarose 1000 (Invitrogen, Inc. cat. # 10975-035), 50× TAE buffer (Invitrogen, Inc. cat. # 24710-030), and ethidium bromide solution (10 mg/ml) (Invitrogen, Inc. cat. # 15585-011). To clone the prepared double-stranded DNA, 0.25 pmole (2.5 μl) cDNA (insert) was used for ligation to pBluescript vector and 0.01788 pmole of pBluescript vector was used to obtain about a 14 to 1 insert to vector ratio. The ligation mixture was prepared by adding to a microfuge tube 1.4 μl of a 1:5 dilution of pBluescript (stock 89.4 nM), 2.5 μl of cDNA, 10 μl of 10× T4 Quick Ligation mix buffer, 6.1 μl of nuclease-free water, and 1 μl of T4 Quick Ligation enzyme in a total reaction volume of 20 μl. The ligation mixture was incubated at room temperature for 10 minutes, then stored at about 4° C. or used immediately.

Transformation of XL1-Blue Electroporation-Competent Cells

Transformation of cells was accomplished using electroporation. Briefly, all reagents were kept on ice and electrocompetent cells were thawed on ice. Ligation reaction (1.5 μl) was added to 40 μl of electrocompetent cells in a microfuge tube and the mixture was transferred to a 0.1 cm Gene Pulser Cuvette (Bio-Rad Laboratories, Hercules, Calif.). Transformation was performed using the following settings on the Gene Pulser; 1700V, 200Ω, and 25 μF. Following electroporation, the cells were transferred to 960 μl of SOC medium and incubated at 37° C. for 1 hour. Cells were then plated onto Brain Heart Infusion agar plates supplemented with ampicillin (100 μg/ml) and incubated at 37° C. overnight.

Plasmid Screening Via Restriction Endonuclease Digestion

To identify cells containing cloned Pegaptanib-derived nucleic acid sequences, randomly picked bacterial colonies were subplated onto Brain Heart Infusion agar plates supplemented with ampicillin (100 μg/ml) and incubated at 37° C. overnight. Individual colonies were picked into 5 ml LB broth supplemented with ampicillin (100 μg/ml) and grown overnight at 37° C. Plasmid DNA was extracted from the bacterial cultures using Qiaprep Spin Miniprep DNA columns as per the manufacturer's instructions.

Plasmid DNA samples were endonuclease digested using XhoI and XbaI to identify the DNA samples containing the double-stranded cDNA inserts (positive clones). Samples were sequenced using a commercial sequencing facility (Harvard Medical DNA Core Sequencing Facility).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the nucleotide sequence of a modified nucleic acid including 2′-modified nucleotides, comprising:
(a) obtaining a sample of a modified nucleic acid comprising a multiplicity of 2′-modified nucleotides,
(b) synthesizing a first cDNA complementary to said modified nucleic acid using a reverse transcriptase;
(c) synthesizing a second cDNA complementary to said first cDNA using a DNA polymerase to form a double-stranded cDNA;
(d) producing multiple double-stranded copies of said double-stranded cDNA; and
(e) sequencing said double-stranded copies;
wherein said modified nucleic acid includes a multiplicity of 2′-fluorinated nucleotides and a multiplicity of 2′-O-methylated nucleotides.

2. The method of claim 1, wherein said 2′-fluorinated nucleotides comprise between 10% and 70% of the total nucleotides in said modified nucleic acid.

3. The method of claim 1, wherein said 2′-fluorinated nucleotides comprise at least 40% of the total nucleotides in said modified nucleic acid.

4. The method of claim 1, wherein said 2′-O-methylated nucleotides comprise between 10% and 70% of the total nucleotides in said modified nucleic acid.

5. The method of claim 1, wherein said 2′-O-methylated nucleotides comprise at least 40% of the total nucleotides in said modified nucleic acid.

6. The method of claim 1, wherein said 2′-fluorinated nucleotides comprise between 10% and 70% of the total nucleotides in said modified nucleic acid and said 2′-O-methylated nucleotides comprise between 10% and 70% of the total nucleotides in said modified nucleic acid.

7. The method of any one of claims 1-6, wherein said modified nucleic acid further comprises a 5′ covalent modification comprising a large hydrophilic moiety.

8. A method for determining the nucleotide sequence of a modified nucleic acid including a 5′ or 3′ large hydrophilic moiety, comprising:
(a) obtaining a sample of a modified nucleic acid comprising a 5′ or 3′ large hydrophilic moiety,
(b) synthesizing a first cDNA complementary to said modified nucleic acid using a reverse transcriptase;
(c) synthesizing a second cDNA complementary to said first cDNA using a DNA polymerase to form a double-stranded cDNA;
(d) producing multiple double-stranded copies of said double-stranded cDNA; and
(e) sequencing said double-stranded copies.

9. The method of claim 8, wherein said large hydrophilic moiety is selected from the group consisting of branched or straight-chain, substituted or unsubstituted, homopolymers or heteropolymers of alkyl, akenyl, aryl, or heterocyclic groups

10. The method of claim 9, wherein said large hydrophilic moiety is a polyethylene glycol moiety.

11. The method of claim 9, wherein said large hydrophilic moiety is a 10-50 kDa polyethylene glycol moiety.

12. The method of any one of claims 8-11, wherein said modified nucleic acid further comprises at least one 2′-fluorinated nucleotide or at least one 2′-O-methylated nucleotide.

13. The method of claim 1, further comprising:
(i) purifying said first cDNA after step (b); and
(ii) polyadenylating the 3′-end of said first cDNA prior to step (c).

14. The method of claim 1, wherein step (d) comprises:
(i) ligating said double-stranded cDNA into a cloning vector;
(ii) transforming host cells with said cloning vector;
(iii) isolating said cloning vector from descendants of said host cells;
(iv) isolating said double-stranded copies from said host cells.

15. The method of claim 1 wherein step (d) comprises performing the polymerase chain reaction using said double-stranded cDNA as an original template molecule.

16. A method of synthesizing a DNA complementary to a modified nucleic acid, the modified nucleic acid comprising 2′-modified nucleotides, the method comprising:
(a) obtaining a sample of a modified nucleic acid comprising a multiplicity of 2′-modified nucleotides,
(b) synthesizing a first cDNA complementary to said modified nucleic acid using a reverse transcriptase,
wherein said modified nucleic acid includes a multiplicity of 2′-fluorinated nucleotides and a multiplicity of 2′-O-methylated nucleotides.

17. The method of claim 16, wherein said modified nucleic acid further comprises a 5′ covalent modification comprising a large hydrophilic moiety.

18. The method of claim 16, wherein said large hydrophilic moiety is selected from the group consisting of branched or straight-chain, substituted or unsubstituted, homopolymers or heteropolymers of alkyl, akenyl, aryl, or heterocyclic groups.

19. The method of claim 18, wherein said large hydrophilic moiety is a polyethylene glycol moiety.

20. The method of claim 18, wherein said large hydrophilic moiety is a 10-50 kDa polyethylene glycol moiety.

21. The method of claim 16, wherein the modified nucleic acid further comprises a modified 3′ terminus.
